SEQUENCE LISTING (2 sequences)

SEQ ID NO: 1 - RNA, 12 nt, Unknown
  Seq. ID. No. 1 is a Eukaryotic Poly A tail. (“Unknown” chosen, since sequence is not an Artificial Sequence, and is not meant to represent genetic material from just one Genus/Species, but rather is common sequence among eukaryotes.)
  polyAsite (1)..(12)
aaaaaaaaaaaa

SEQ ID NO: 2 - DNA, 12 bp, Artificial Sequence
  Chemically synthesized (Poly T for hybridization to Poly A tail)
tttttttttttt

(19) United States
(12) Patent Application Publication
(10) Pub. No.: US 2008/0033159 A1
(43) Pub. Date: Feb. 7, 2008

(54) Method for small volume nucleic acid synthesis

(76) Inventor: James M. Kadushin, Gilbertsville, PA (US)

Correspondence Address: LAW OFFICE OF MORRIS E. COHEN, 1122 CONEY ISLAND AVENUE, SUITE 217, BROOKLYN, NY, 11230, US

(21) Appl. No.: 11/584,751

(22) Filed: Oct. 20, 2006

Related U.S. Application Data
(63) Continuation of application No. 11/388,772, filed on Mar. 24, 2006, now abandoned.
Application No. 11/388,772 is a continuation of application No. PCT/US04/31804, filed on Sep. 27, 2004.
Application No. 11/388,772 is a continuation-in-part of application No. 10/825,776, filed on Apr. 16, 2004, now abandoned.
Application No. 10/825,776 is a continuation of application No. 10/050,088, filed on Jan. 14, 2002, now abandoned.
Application No. PCT/US04/31804 is a continuation-in-part of application No. 10/234,069, filed on Sep. 3, 2002.
Application No. PCT/US04/31804 is a continuation-in-part of application No. PCT/US03/09232, filed on Mar. 25, 2003.
(60) Provisional application No. 60/506,247, filed on Sep. 26, 2003.
(60) Provisional application No. 60/261,231, filed on Jan. 13, 2001.
(60) Provisional application No. 60/316,116, filed on Aug. 31, 2001.
(60) Provisional application No. 60/367,438, filed on Mar. 25, 2002.

(51) Int. Cl. C07H 21/00

(52) U.S. Cl. 536/25.32

(57) ABSTRACT

A method for synthesis of nucleic acids, with particular use for synthesis of cDNA. The method allows application of the cDNA to a microarray without the need for concentration or purification of the cDNA post-cDNA synthesis.

### RELATED APPLICATIONS

The present application is a continuation of U.S. Nonprovisional application Ser. No. 11/388,772 filed Mar. 24, 2006, which is a continuation of PCT Application Serial No. PCT/US2004/031804 (“the '804 application”) filed Sep. 27, 2004, which claims the priority of U.S. Provisional Application Ser. No. 60/506,247 filed Sep. 26, 2003.

The '804 application is also a continuation-in-part of U.S. Nonprovisional application Ser. No. 10/825,776 filed Apr. 16, 2004 (pending), which is a continuation of U.S. Nonprovisional application Ser. No. 10/050,088 filed Jan. 14, 2002 (abandoned), which claims the priority of U.S. Provisional Application Ser. No. 60/261,231 filed Jan. 13, 2001.

The '804 application is also a continuation-in-part of U.S. Nonprovisional application Ser. No. 10/234,069 filed Sep. 3, 2002 (pending), which claims the priority of U.S. Provisional Application Ser. No. 60/316,116 filed Aug. 31, 2001.

The '804 application is also a continuation-in-part of PCT Application Serial No. PCT/US03/09232 filed Mar. 25, 2003 (pending) (“the '232 application”) which claims the priority of U.S. Provisional Application Ser. No. 60/367,438 filed Mar. 25, 2002.

The priority of all of those applications is claimed, all of which are fully incorporated herein by reference.

### FIELD OF THE INVENTION

The present invention relates to an improved method for nucleic acid synthesis.

### BACKGROUND OF THE INVENTION

The synthesis of nucleic acids is one of the cornerstones of modern molecular biology, and is utilized in a large variety of experimental and diagnostic techniques. Of the various forms of nucleic acids currently known, cDNA or “Complementary DNA” is a DNA copy made off of a messenger RNA (mRNA) or other type of RNA molecule template.

Synthesis of the cDNA molecule from the original mRNA is accomplished using an enzyme known as Reverse Transcriptase (RT), which is an RNA dependent DNA polymerase. Reverse Transcriptase was initially discovered in connection with retroviruses, and can be obtained via purification from such a virus, such as avian myoblastosis virus (AMV) or Moloney murine leukemia virus (M-MuLV), or from a cell containing the cloned gene.

Using the mRNA isolated from a given cellular source, a library can be constructed of cDNA molecules complementary to the cellular mRNA. That cDNA library can then be used for various experimental purposes. For example, a cDNA library formed from a particular tissue type can used for gene expression analysis, i.e. to provide information regarding the expression of nucleic acids in the initial sample. Gene expression analysis may be of use in a variety of applications, including, for example, the identification of novel expression of genes, the correlation of gene expression to a particular phenotype, screening for disease predisposition, and identifying the effect of a particular agent on cellular gene expression, such as in toxicity testing and screening for new drug compounds.

To perform an analysis, total (or messenger) RNA is extracted from the desired sample of cells. Copies of complementary DNA (cDNA) are generated from the RNA through reverse transcription. The cDNA copies are tagged with a marker or label such as a fluorescent marker and broken up into short fragments.

As discussed in prior patent applications of the present inventor, analysis of the cDNA sequences in a given sample can be particularly efficient when conducted using high-speed technologies for nucleic acid analysis such as a DNA microarray (see e.g. U.S. Nonprovisional application Ser. No. 10/234,069 filed Sep. 3, 2002 which claims the priority of U.S. Provisional Application Ser. No. 60/316,116 filed Aug. 31, 2001, and “Methods for Blocking Nonspecific Hybridizations of Nucleic Acids”, International Application No. PCT/US02/027799 filed 3 Sep. 2002, International Publication No. WO 03/020902 A2, all of which are fully incorporated herein by reference). All microarrays operate on a similar principle: a substantially planar substrate such as a glass slide or a silicon chip or nylon membrane is coated with a grid of tiny spots of about 20 to 100 microns in diameter. Each spot (i.e. feature) contains millions of copies of a short sequence of DNA or nucleotides; a computer keeps track of the location of each sequence on the substrate, allowing the user to conduct thousands of miniature test-tube like reactions simultaneously.

After tagging of the cDNA copies, the tagged fragments are washed over the microarray and left overnight, to allow the tagged fragments to hybridize with the DNA attached to the microarray. After hybridization, the features on the microarray that have paired with the fluorescent cDNA emit a fluorescent signal that can be viewed with a microscope or detected by a computer. In this manner, one can learn which sequences on the microarray match the cDNA of the test sample. Although there are occasional mismatches, the employment of millions of probes in each spot or feature ensure fluorescence is detected only if the complementary cDNA is present. The more intense the fluorescent signal, (i.e. the brighter the spot) the more matching cDNA was present in the cell.

Unfortunately, however, in the traditional methods known in the art, after preparation of the cDNA through reverse transcription, concentration of the cDNA must be conducted before the cDNA can be used in the hybridization mix applied to the microarray. Often purification must be conducted as well, such as when the cDNA has been labelled using dyes. These concentration and/or purification steps add additional time and expense to the experimental analysis.

One typical method for nucleic acid concentration, for example, is ethanol precipitation. An illustrative protocol for ethanol precipitation to enable cDNA concentration is as follows:

### Ethanol Precipitation of Synthesized cDNA

1. Adjust the volume of synthesized cDNA to 130 uL with 1×TE buffer.
2. Add 3 μl of the linear acrylamide (5.0 mg/mL) to the synthesized cDNA mix.
3. Add 6 μl of 5M NaCl or 250 μl 3M Ammonium Acetate and mix.
4. Add 540 μl of 100% ethanol if using NaCl or 875 μl of 100% ethanol if using 3M Ammonium Acetate. Mix by moderate vortexing.
5. Incubate at −20 degrees Celsius for 30 minutes.
6. Centrifuge the sample at >10,000 g for 15 minutes.
7. Carefully aspirate the supernatant to avoid loss of the cDNA pellet. Do not decant, as decanting may dislodge the pellet and cause it to be lost.
8. Add 300 μl of 70% ethanol to the cDNA pellet. Gently mix by tapping the side of the tube. Avoid overmixing, which may cause the cDNA pellet to break up.
9. Centrifuge at >10,000 g for 5 minutes and remove the supernatant. Do not decant.
10. Dry the cDNA pellet completely by heating for 10-30 minutes at 65 degrees Celsius. If the cDNA pellet is not completely dry, it will be difficult to resuspend, and incomplete resuspension may produce high speckled background on the microarray and/or weak results.
11. Proceed to hybridization of the cDNA to the array.

Although ethanol precipitation is a traditional and accepted method for nucleic acid concentration, unfortunately, it may lead to variable results due to partial or complete loss of the pelleted cDNA or incomplete re-solubilization of the precipitated cDNA. For example, as can be seen above, problems may occur if the ethanol precipitation procedure is not performed carefully because reverse transcription of small quantities of RNA produces a cDNA pellet that is very small and easily lost during processing or by adherance to the inside of pipet tips.

An alternate method for concentration of the cDNA pellet is concentration with Microcon® microconcentrators. A sample protocol for Microcon® concentration is as follows:

### Concentration of cDNA with Millipore Microcon® YM-30 Centrifugal Filter Devices

cDNA samples may be concentrated using the Millipore Microcon® YM-30 Centrifugal Filter Devices (30,000 molecular weight cutoff, Millipore catalog number 42409). The following protocol is an example of a method provided to reduce the volume of the cDNA synthesis reaction from 130 μl to 3-10 μl, for hybridization to an array. (Note: while the following sample protocol is similar to that provided by the manufacturer, it includes minor modifications for use with the 3DNA Array 350 kit. Additionally, users of the Microcon YM-30 should evaluate their own centrifuge settings to determine the optimal time and speed settings to yield final volumes of 3-10 μl).
1. Place the Microcon® YM-30 sample reservoir into the 1.5 ml collection tube.
2. Pre-wash the reservoir membrane by adding 100 μl TE pH 8.0 to the Microcon® YM-30 sample reservoir.
3. Place the tube/sample reservoir assembly into a fixed angle rotor tabletop centrifuge capable of 10-14,000 g.
4. Spin for 3 minutes at 10-14,000 g.
5. Add all 130 μl from the cDNA reaction to the Microcon® YM-30 sample reservoir. Do not touch the membrane with the pipet tip.
6. Place the tube/sample reservoir assembly into a fixed angle rotor tabletop centrifuge capable of 10-14,000 g.
7. Centrifuge for 8-10 minutes at 10-14,000 g.
8. Remove the tube/sample reservoir assembly. Separate the collection tube from the sample reservoir with care, avoiding spilling any liquid in the sample reservoir.
9. Add 5 μl of 1×TE buffer (10 mM Tris-HCl, pH 8.0/1 mM EDTA) to the sample reservoir membrane without touching the membrane. Gently tap the side of the concentrator to promote mixing of the concentrate with the 1×TE buffer.
10. Carefully place the sample reservoir upside down on a new collection tube. Centrifuge for 2 minutes at top speed in the same centrifuge.
11. Separate the sample reservoir from the collection tube and discard the reservoir. Note the volume collected in the bottom of the tube (3-10 μl total volume). The cDNA sample may be stored in the collection tube for later use.
12. Proceed to hybridization of the cDNA to the array.

As can be seen from the above examples, the sample concentration protocols traditionally utilized prior to application of the cDNA to a microarray require a time consuming extra series of steps after cDNA synthesis. In some cases, these additional steps can decrease performance and the results obtained in the assay.

Accordingly, it is an object of the present invention to provide an improved method for cDNA synthesis which eliminates the need for the post-synthesis sample concentration discussed above.

### SUMMARY OF THE INVENTION

It is an object of the present invention to provide an improved method for nucleic acid synthesis.

It is a further object of the present invention to provide an improved method for synthesis of cDNA.

It is a further object of the present invention to provide a method which avoids the need for concentration of the cDNA after cDNA synthesis.

It is a further object of the present invention to provide a method which avoids the need for a purification step to purify undesirable molecules from the cDNA sample after cDNA synthesis.

It is a further object of the present invention to provide a method for synthesis of cDNA using very small quantities of sample materials.

It is a further object of the present invention to provide a method for synthesis of cDNA which provides increased sensitivity.

It is a further object of the invention to provide an improved method for cDNA synthesis for use with microarrays.

It is a further object of the present invention to provide a method for synthesis of cDNA, which allows application of the cDNA to a microarray without the need for concentration of the cDNA after cDNA synthesis.

It is a further object of the present invention to provide a method for synthesis of cDNA, which allows application of the cDNA to a microarray without the need for purification of undesirable molecules from the cDNA sample after cDNA synthesis.

It is a further object of the present invention to provide a method for synthesis of cDNA for application to microarrays in a readily automatable fashion.

Further to the above objects, in accordance with the present invention, a method is disclosed for improved nucleic acid synthesis. The method provides higher sensitivity results from very small quantities of sample materials than provided by traditional methods in the art by eliminating a sample concentration protocol after nucleic acid synthesis.

In the preferred embodiments, the invention is used for the synthesis of cDNA (complementary DNA) from an initial RNA samples, such as mRNA from a source of interest. In further preferred embodiments, the method is used for nucleic acids intended for application to microarrays, for assay of those nucleic acids using hybridization of the synthesized nucleic acids to known molecules affixed to the array. In further preferred embodiments, the assays are conducted using dendritic nucleic acid reagents. Yet further preferably, capture sequences are used to label the synthesized nucleic acids and/or the nucleic acids on the array.

In further embodiments of the invention, kits may be provided for conducting the methods disclosed herein. Additionally, the processes can also be used for other types of sample preparations for unrelated applications.

The avoidance of the need for a post-synthesis sample concentration protocol is a significant advantage of the present method, particularly since such protocols can cause excessive loss of sample. The method is particularly useful with small quantity preparations starting with less than one microgram (1000 nanograms) of total RNA or equivalent nucleic acid sample.

A further significant advantage of the present invention is that it provides a reduction of the time and number of operations required to perform complete cDNA synthesis. This advantage is of particular importance for a variety of contexts and applications, including the needs of research laboratories, diagnostic kits, clinical settings, and so forth.

Yet a further significant advantage of the invention is that it provides a better consistency of final cDNA yield based on the same input materials, leading to better reproducibility of results.

Further objects and advantages of the invention will become apparent in conjunction with the detailed disclosure provided herein.

### BRIEF DESCRIPTION OF THE DRAWINGS

FIG. 1 is a schematic view of a preferred method in accordance with the present invention.

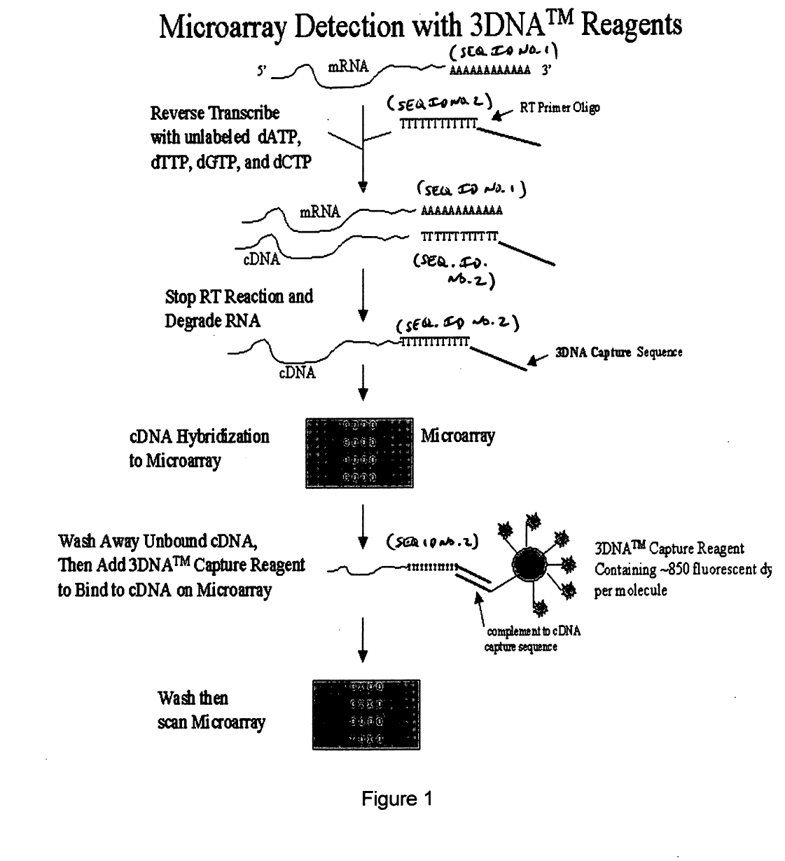

### DETAILED DESCRIPTION OF THE INVENTION AND THE PREFERRED EMBODIMENTS

Before the present invention is further described, it is to be understood that the invention is not limited to the particular embodiments of the invention described herein, as variations of the particular embodiments may be made and still fall within the scope of the invention or the appended claims. It is also to be understood that the terminology employed is for the purpose of describing particular embodiments, and is not intended to be limiting.

The present invention is generally directed to a method for provided for nucleic acid synthesis which avoids the need for post-synthesis sample concentration. The cost effective and efficient manner by which the nucleic acid sequence samples are prepared and by which the methods of the present invention are implemented, using conventional laboratory techniques, equipment and reagents, make them especially suitable for research and clinical use, and for automation.

The invention is particularly suitable for nucleic acid synthesis, such as cDNA synthesis, conducted in conjunction with assay on a microarray. In such methods, an array of DNA or gene probes fixed or stably associated with the surface of a substantially planar substrate (“a microarray”) is contacted with a sample of target nucleic acids under hybridization conditions sufficient to produce a hybridization pattern of complementary probe/target complexes. A variety of different microarrays which may be used are known in the art. The hybridized samples of nucleic acids are then targeted by labeled probes and hybridized to produce a detectable signal corresponding to a particular hybridization pattern. The individual labeled probes hybridized to the target nucleic acids are all capable of generating the same signal of known intensity. Thus, each positive signal in the microarray can be “counted” in order to obtain quantitative information about the genetic profile of the target nucleic acid sample.

The DNA or gene probes of the microarrays which are capable of sequence specific hybridization with target nucleic acid may be polynucleotides or hybridizing analogues or mimetics thereof, including, but not limited to, nucleic acids in which the phosphodiester linkage has been replaced with a substitute linkage group, such as phophorothioate, methylimino, methylphosphonate, phosphoramidate, guanidine and the like, nucleic acids in which the ribose subunit has been substituted, e.g. hexose phosphodiester; peptide nucleic acids, and the like. The length of the probes will generally range from 10 to 1000 nucleotides, although the present invention is not limited to probes of such lengths. In some embodiments of the invention, for example, the probes will be oligonucleotides having from 15 to 150 nucleotides and more usually from 15 to 100 nucleotides. In other embodiments the probes will be longer, usually ranging in length from 150 to 1000 nucleotides, where the polynucleotide probes may be single or double stranded, usually single stranded, and may be PCR fragments amplified from cDNA. The DNA or gene probes on the surface of the substrates will preferably correspond to known genes of the physiological source being analyzed and be positioned on the microarray at a known location so that positive hybridization events may be correlated to expression of a particular gene in the physiological source from which the target nucleic acid sample is derived. Because of the manner in which the target nucleic acid sample is generated, as described below, the microarrays of gene probes will generally have sequences that are complementary to the non-template strands of the gene to which they correspond.

The substrates with which the gene probes are stably associated may be fabricated from a variety of materials, including plastic, ceramic, metal, gel, membrane, glass, and the like. The microarrays may be produced according to any convenient and conventional methodology, such as preforming the gene probes and then stably associating them with the surface of the support or growing the gene probes directly on the support. A number of different microarray configurations and methods for their production are known to those of skill in the art, one of which is described in Science, 283, 83, 1999, the content of which is incorporated herein by reference.

In accordance with the method of the present invention, a desired microarray is provided having the probe nucleic acid sequences stably affixed thereto. In addition, a sample is provided having the target molecules of interest for study. The target molecules are labelled for detection, the term “label” is used herein to refer to agents that are capable of providing a detectable signal, either directly or through interaction with one or more additional members of a signal producing system. The label is one which preferably does not provide a variable signal, but instead provides a constant and reproducible signal over a given period of time. The target molecules can be labelled prior to or after application of target to the array, although prior labelling is generally preferred. Preferably a very sensitive signal generating method is used, such as a dendrimer or another comparably sensitive signal generating methods, e.g. relative light scatter detection using nanogold labels, such as those of Genicon Inc./Invitrogen.

In conjunction with the present inventions, dendritic nucleic acid molecules are particularly preferred for their detection capabilities (although any type of labelled molecules of suitable sensitivity can be utilized with the inventions disclosed herein). Dendritic nucleic acid molecules, or dendrimers are complex, highly branched molecules, comprised of a plurality of interconnected natural or synthetic monomeric subunits of double-stranded DNA. Dendrimers are described in greater detail in Nilsen et al., Dendritic Nucleic Acid Structures, J. Theor. Biol., 187, 273-284 (1997); in Stears et al., A Novel, Sensitive Detection System for High-Density Microarrays Using Dendrimer Technology, Physiol. Genomics, 3: 93-99 (2000); and in various U.S. patents, such as U.S. Pat. Nos. 5,175,270; 5,484,904; 5,487,973; 6,072,043; 6,110,687; and 6,117,631; all of which are fully incorporated herein by reference. Likewise, various inventions relating to dendrimers and their use on microarrays are described in PCT Application Serial No. PCT/US03/09232 filed 25 Mar. 2003, U.S. Provisional Application Ser. No. 60/367,438 filed Mar. 25, 2002, U.S. Nonprovisional application Ser. No. 10/825,776 filed Apr. 16, 2004, U.S. Nonprovisional application Ser. No. 10/050,088 filed Jan. 14, 2002; U.S. Provisional Application Ser. No. 60/261,231 filed Jan. 13, 2001; U.S. Nonprovisional application Ser. No. 10/730,823 filed Dec. 8, 2003; U.S. Nonprovisional application Ser. No. 10/393,519 filed Mar. 20, 2003; PCT Application Serial No. PCT/US01/29589 filed Sep. 20, 2001; U.S. Provisional Application Ser. No. 60/234,060 filed Sep. 20, 2000; PCT Application Serial No. PCT/US01/29589 filed Sep. 20, 2001; U.S. Nonprovisional application Ser. No. 09/908,950 filed Jul. 19, 2001; U.S. Provisional Application Ser. No. 60/219,397, filed Jul. 19, 2000; U.S. Provisional Application Ser. No. 60/187,681 filed Mar. 8, 2000; U.S. Nonprovisional application Ser. No. 09/802,162 filed Mar. 8, 2001; and U.S. Provisional Application Ser. No. 60/187,681 filed Mar. 8, 2000; PCT Application Serial No. PCT/US2003/009232; and PCT Application No. PCT/US2003/025865; all of which are fully incorporated herein by reference.

Dendrimers comprise two types of single-stranded hybridization “arms” on the surface which are used to attach two key functionalities. A single dendrimer molecule may have at least one hundred arms of each type on the surface. One type of arm is used for attachment of a specific targeting molecule to establish target specificity and the other is used for attachment of a label or marker. The molecules that determine the target and labeling specificities of the dendrimer are attached either as oligonucleotides or as oligonucleotide conjugates. Using simple DNA labeling, hybridization, and ligation reactions, a dendrimer molecule may be configured to act as a highly labeled, target specific probe.

The prepared mixture is formulated in the presence of a suitable buffer to yield a dendrimer hybridization mixture containing dendrimers with fluorescent labels attached to one type of “arm”, and with oligonucleotides attached to another type of “arm”, complementary to the capture sequences of the RT primer bound cDNA fragments. An oligonucleotide designed to block non-specific interaction of the cDNA or the dendrimer to the nucleic acid spotted on the array surface can also be added at this time; blocking oligonucleotides containing the multiplicities of the same nucleic acid base may be used for blocking long stretches of the same complementary base found on the cDNA derived from the RNA sample and the nucleic acid probes on the microarray surface.

To prepare fluorescent labeled dendrimer, the complementary sequences to the capture sequence on the Cy3® RT primer and the Cy5® RT primer are ligated, separately, to the purified dendritic core material as prepared by the previously described methods (see Nilson et al., supra, and U.S. Pat. Nos. '270, '904, and '973, supra.). Thirty nucleotide long oligonucleotides complementary to the outer arms of a four-layer dendrimer having a 5′ Cy3® or Cy5® are then synthesized. (Oligos etc., Inc., Wilsonville, Oreg.). The Cy3® and Cy5® oligonucleotides are then hybridized and covalently cross-linked to the outer surface of the corresponding dendrimers, respectively. Excess capture and fluorescent labeled oligonucleotides are then removed through techniques such as size exclusion chromatography and density gradient ultracentrifugation.

The concentration of dendrimer is determined by measuring the optical density of the purified material at 260 nm on a UV/Vis spectrometer. The fluorescence is measured at optimal signal/noise wavelengths using a fluorometer (FluoroMax, SPEX Industries). Cy3 is excitable at 542 nm and the emission measured at 570 nm. Cy5 is excitable at 641 nm and the emission at 676 nm.

In the preferred embodiments of the present invention, a dendrimer is utilized having approximately 850 fluorescent dyes on each molecule, such as the dendrimers available in the Genisphere, Inc. 3DNA Array 900 labeling kit. Alternatively, prior dendrimers having approximately three hundred dyes can be utilized, or dendrimers having more than 500 fluorescent dyes.

The use of such dendrimer probes significantly increases increasing sensitivity due to the dendrimers' superior signal amplification capability. By increasing sensitivity, the amount of RNA required for an assay is reduced, allowing the use of smaller volumes of initial sample. In particular, the present invention can be easily conducted using 0.25-1 microgram of total RNA, or, as low as 100 nanograms of total RNA (0.1 micrograms); or with 1 to 1000 nanograms of poly A RNA (mRNA). Accordingly, a dendrimer having approximately 850 or more fluorescent dyes, is preferably utilized to obtain these improved results. As the sensitivity of detection improves in the art (e.g. using improved dendrimers or other improved labelling and signal molecules), smaller RNA samples may be assayed using the present invention as well.

For the assay itself, a strategy is preferably used (a “two step method”) that employs successive hybridization steps where the reverse transcribed cDNA is applied to the array for a sufficiently long period to allow hybridization thereto, with hybridization of the cDNA molecules to target immobilized probes being followed by a washing procedure where the unbound cDNA and excess RT primer is removed from the array. The cDNA preferably has a capture sequence incorporated thereto for binding to a dendritic nucleic acid having a label capable of generating a detectable signal, as disclosed below. The fluorescently labeled dendrimer molecule (or another molecule capable of binding to the capture sequence incorporated into the cDNA) is subsequently applied to the washed array and hybridizes to the cDNA associated capture sequence during this second hybridization. Excess dendrimer is washed away during a secondary washing procedure and the arrays are scanned to detect signal generated by the label molecule.

If desired, in further preferred embodiments, temperature cycling can be used to selectively control hybridization between the target nucleic acid and the microarray, and hybridization between the capture reagent and the microarray (preferably cDNA—microarray hybridization and cDNA—dendrimer hybridization, respectively). By using such cycling, hybridization can be carefully controlled such that cDNA initially hybridizes only to the microarray, with subsequent hybridization of cDNA to the dendrimer. This procedure can be used to improve the kinetics of hybridization of each of the two components, i.e. target nucleic acid to probe, and capture reagent to target nucleic acid. Further details regarding use of such temperature cycling are provided in U.S. Nonprovisional application Ser. No. 10/050,088 filed on Jan. 14, 2001, U.S. Provisional Application No. 60/261,231 filed Jan. 13, 2001, and in published protocols by the present inventors and by Genisphere, Inc. of Montvale, N.J., all of which are fully incorporated herein by reference.

In the preferred embodiments of the present invention, the nucleic acid synthesized will generally be DNA that has been reverse transcribed from RNA derived from a naturally occurring source, where the RNA may be selected from the group consisting of total RNA, poly(A)⁺ RNA, amplified RNA and the like. The initial RNA source may be present in a variety of different samples, where the sample will typically be derived from a physiological source. The physiological source may be derived from a variety of sources, with physiological sources of interest including sources derived from single celled organisms such as yeast or bacteria, and multicellular organisms, including plants and animals, particularly mammals, where the physiological sources from multicellular organisms may be derived from particular organs or tissues of the multicellular organism, or from isolated cells derived therefrom. In obtaining the sample RNAs to be analyzed from the physiological source from which it is derived, the physiological source may be subjected to a number of different processing steps, where such known processing steps may include tissue homogenation, cell isolation and cytoplasmic extraction, nucleic acid extraction, poly A tailing, and the like. Methods of isolating RNA from cells, tissues, organs or whole organisms are known to those of ordinary skill in the art and are described, for example, in Maniatis et al., Molecular Cloning: A Laboratory Manual, 2^{nd} ed., Cold Spring Harbor Laboratory Press, 1989, and in Ausubel et al., Current Protocols in Molecular Biology, John Wiley & Sons, Inc., 1998, both of which are fully incorporated herein by reference.

The sample mRNA is preferably reverse transcribed into a target nucleic acid in the form of a cDNA, by hybridizing an oligo(dT) primer, or RT primer, to the mRNA under conditions sufficient for enzymatic extension of the hybridized primer. The primer will be sufficiently long to provide for efficient hybridization to the mRNA tail, where the region will typically range in length from 10 to 25 nucleotides, usually 10 to 20 nucleotides, and more usually from 12 to 18 nucleotides.

Recognizing that applications typically require the use of sequence specific primers, the standard primers as used in the present invention further include “capture sequence” nucleotide portions. The preferred capture sequences referred to herein are Cy3® RT primer capture sequences (Oligos etc., Inc, Wilsonville, Oreg.) or Cy5® RT primer capture sequence (Oligos etc., Inc, Wilsonville, Oreg.), as disclosed for example in International Application No. PCT/US02/027799 filed 3 Sep. 2002, which is fully incorporated herein by reference.

For custom primers, the capture sequences should be attached to the 5′ end of the corresponding custom oligonucleotide primer. In this manner, the custom primer replaces the standard RT primer. Since the present invention is devised for use with the standard RT primer, some modifications may be required when substituting a custom primer. Such modifications are known to those of ordinary skill in the art and may include adjusting the amount and mixture of primers based on the amount and type of RNA sample used. The primer carries a capture sequence comprised of a specific sequence of nucleotides, as described above. The capture sequence is complementary to the oligonucleotides attached to the arms of dendrimer probes which further carry at least one label. Such complementary oligonucleotides may be acquired from any outside vendor and may also be acquired as labeled moieties. The label may be attached to one or more of the oligonucleotides attached to the arms of the dendrimer probe, either directly or through a linking group, as is known in the art. In the preferred embodiment, the dendrimer probes are labeled by hybridizing and cross-linking Cy3® or Cy5® labeled oligonucleotides to the dendrimer arms. The Cy3® or Cy5® labeled oligonucleotides are complementary to the Cy3® or Cy5® RT primer capture sequences, respectively.

In generating the target nucleic acid sample, the primer is contacted with the RNA in the presence of a reverse transcriptase enzyme, and other reagents necessary for primer extension under conditions sufficient for inducing first strand cDNA synthesis. A variety of enzymes, usually DNA polymerases, possessing reverse transcriptase activity can be used for the first strand cDNA synthesis step. Examples of suitable DNA polymerases include the DNA polymerases derived from organisms selected from the group consisting of a thermophilic bacteria and archaebacteria, retroviruses, yeasts, Neurosporas, Drosophilas, primates and rodents. Suitable DNA polymerases possessing reverse transcriptase activity may be isolated from an organism, obtained commercially or obtained from cells which express high levels of cloned genes encoding the polymerases by methods known to those of skill in the art, where the particular manner of obtaining the polymerase will be chosen based primarily on factors such as convenience, cost, availability and the like. The order in which the reagents are combined may be modified as desired.

In one preferred embodiment of the invention, the cDNA synthesis protocol involves combining total RNA or poly(A)⁺ RNA (mRNA), with RT primer and RNase free water to yield the RNA-RT primer mix. In accordance with the present invention, very small quantities of initial RNA can be utilized, as discussed below.

The RNA-RT primer mixture is then mixed and microfuged to collect the contents at the bottom of the microfuge tube. The RNA-RT primer mixture is then heated at a suitable temperature (e.g. 80 degrees Celsius) for ten minutes and immediately transferred to ice. In a separate microfuge tube on ice, RT buffer, DTT (dithiothreitol), RNAse inhibitor, dNTP mix, and reverse transcriptase enzyme are combined with RNase free water (the Reaction Master Mix). This combination is gently mixed (but not vortexed) and microfuged briefly to collect the contents at the bottom of the microfuge tube to yield a reaction mixture. The RNA-RT primer mixture is then mixed with the Reaction Master Mix and incubated at a suitable temperature (e.g. about 42 degrees Celsius) for a period of time sufficient for forming the first strand cDNA primer extension product, which usually takes about 2 hours. The reaction is stopped using 1.0M NaOH/100 mM EDTA, and then incubated at a suitable temperature (e.g. 65 degrees Celsius) to denature the DNA/RNA hybrids and degrade the RNA. The reaction is then neutralized with 2M Tris-HCl (pH 7.5).

Once this is completed, the cDNA is applied directly to the array for hybridization thereto without post-synthesis concentration of the cDNA. (In an additional embodiment, there is no post-synthesis purification conducted of the cDNA, either). Rather, the mixture obtained above is directly added to the microarray and incubated at a second hybridization temperature and for a sufficient time to allow the cDNA to bind to the microarray. Suitable hybridization conditions are well known to those of skill in the art and reviewed in Maniatis et al., supra, where conditions may be modulated to achieve a desire specificity in hybridization. If desired, a blocking LNA oligonucleotide can be used to reduce non-specific binding between the cDNA and the array, as discussed in International Application No. PCT/US02/027799 filed 3 Sep. 2002, International Publication No. WO 03/020902 A2, which is fully incorporated herein by reference. The array is then washed to reduced background on the array (e.g. caused by free RT primer not incorporated into cDNA molecules).

A label molecule (preferably a dendrimer carrying a desired label) is then applied to the array for hybridization of the label to the capture sequence of the cDNA to provide a detectable signal. Following the hybridization step, a washing step is employed in which unhybridized complexes are purged from the microarray, thus leaving behind a visible, discrete pattern of hybridized cDNA-dendrimer probes bound to the microarray. A variety of wash solutions and protocols for their use are known to those of skill in the art and may be used. The specific wash conditions employed will necessarily depend on the specific nature of the signal producing system that is employed, and will be known to those of skill in the art familiar with the particular signal producing system employed.

The resultant hybridization pattern of labeled cDNA fragments may be visualized or detected in a variety of ways, with the particular manner of detection being chosen based on the particular label of the cDNA, where representative detection means include scintillation counting, autoradiography, fluorescence measurement, calorimetric measurement, light emission measurement and the like.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured.

Following detection or visualization, the hybridization pattern can be used to determine quantitative and qualitative information about the initial RNA sample. For example, information can be obtained regarding the genetic profile of the labeled target nucleic acid sample that was contacted with the microarray to generate the hybridization pattern. From this data, one can also derive information about the physiological source from which the target nucleic acid sample was derived, such as the types of genes expressed in the tissue or cell which is the physiological source, as well as the levels of expression of each gene, particularly in quantitative terms. Where one uses the subject methods in comparing target nucleic acids from two or more physiological sources, the hybridization patterns may be compared to identify differences between the patterns. Where microarrays in which each of the different probes corresponds to a known gene are employed, any discrepancies can be related to a differential expression of a particular gene in the physiological sources being compared. Thus, for example, the subject methods find use in differential gene expression assays, where one may use the subject methods in the differential expression analysis of: diseased and normal tissue, e.g. neoplastic and normal tissue, different tissue or subtissue types; and the like.

Many other variations of the above procedures can be used consistent with the present invention. For example, instead of utilizing RNA extracted from a sample which is converted to cDNA prior to hybridization, the present invention can be used with the RNA sample directly. In one such embodiment, a suitable capture sequence can be ligated to the RNA using known methods of splicing RNA, such as through enzymatic means. Or, if the RNA includes a specific oligonucleotide that is useful as a capture sequence, a complementary oligonucleotide can be attached to a dendrimer to label the RNA molecule. Further details regarding methods for direct use of RNA without the need for reverse transcription are provided in PCT Application No. PCT/US01/22818 filed Jul. 19, 2001, which is fully incorporated herein by reference.

The invention is particularly suitable as for improved synthesis and assay of cDNA using very small quantities of initial starting materials, as the present invention allows the use of less initial RNA sample than prior methods. For example, when partnered with the 3DNA dendrimer technology (or other very sensitive signal generating methods, e.g. relative light scatter detection using nanogold labels, such as those of Genicon Inc./Invitrogen), this cDNA method is particularly valuable as it allows for use of total RNA samples down to 250 nanograms or less without any need for RNA target amplification. The invention can be easily used with 0.25-1 microgram of sample, or, if desired with 100 nanograms of initial total RNA sample (0.1 micrograms); or with 1 to 1000 nanograms of poly A RNA (mRNA). This is about 2 orders of magnitude better than direct incorporation methods, which require 20-50 micrograms of total RNA, or 1 to 2.5 micrograms of poly A RNA (mRNA) for the same level of sensitivity.

The present methods, therefore, allow for use of very small starting samples that otherwise would be lost during the post-synthesis concentration or purification steps. As the sensitivity of detection increases in the art (e.g. using improved dendrimers, or dendrimers having additional fluorescent dyes, or other improved signal generating methods), smaller samples of RNA can be utilized in conjunction with the present invention as well. Those samples can likewise be applied to the microarray without concentration of the cDNA post-synthesis.

The invention provides the ability to scale up the reaction and yet still avoid the need for concentration of purification of the final cDNA synthesis. A further benefit of the invention is the reduction of non-specific background on microarrays caused directly or indirectly by the presence of high amounts of cDNA, which are avoided by using this method. Likewise, the method provides increased data quality (specifically, the range of differentials in a gene expression experiment), which is likely due to the very small quantity of cDNA used in the array hybridization assay. This is due to the fact that, as opposed to prior methods, in the present invention no concentration is conducted of the cDNA post synthesis. Previous methods generally require concentration of cDNA for using the cDNA on most types of arrays due to the larger sample size required with non-3DNA methods and the small volumes required for the microarray hybridization.

Likewise, a further improvement provided herein is that the present method does not require purification of the cDNA synthesis reaction, which is normally performed for direct dye incorporation cDNA synthesis to remove excess dye and enzyme not incorporated into the cDNA.

In addition, the present invention provides the further advantage of being readily automatable, as opposed to the concentration and purification steps normally used in this context, which are currently difficult to accomplish in an automated fashion or robotically.

Further advantages, features and aspects of the present invention will be apparent in conjunction with the following examples.

### Example 1

### cDNA Synthesis from Total RNA

1. In a microtube combine:
   1-5 μl total RNA (0.25-1.0 g mammalian total RNA or 0.5-2.5 μg plant total RNA)
   1 μl RT Primer (5 pmole/μl)
   Add Nuclease Free Water to a final volume of 6 μl
   This is the RNA-RT primer mix.
   Note: the use of 5 pmole/μl RT Primer may cause non-specific background signal on certain types of arrays (i.e. poly-L-lysine coated slides). This type of background may be reduced by diluting the RT Primer by up to 2.5 fold with Nuclease Free Water prior to its addition to the RNA sample (caution: avoid diluting the primer to less than 2 pmole/μl as this may cause inefficient cDNA synthesis).
2. Mix and microfuge briefly to collect contents in the bottom of the tube.
3. Heat to 80° C. for five minutes and immediately transfer to ice for 2-3 minutes.
4. When provided as part of a kit, Reverse transcriptase enzyme and the enzyme's reaction buffer can be included within that kit or can be purchased separately. The use of SuperScript IT Reverse Transcriptase Enzyme (Gibco Cat No. 18064-014-10,000 Units @ 200U/μl) is recommended.
   The Reaction Master Mix should be formulated to a final volume dependent on the number of cDNA syntheses set up simultaneously. Each cDNA synthesis requires 4.5 μL of Reaction Master Mix. To reduce pipetting errors, the Reaction Master Mix should contain at least 9 μL (enough for two cDNA syntheses).

Combine on ice in a separate microtube according to the chart below:

| Number of cDNA synthesis | 1 | 2 | 3 | 4 | 5 | 10. . . |
|---|---|---|---|---|---|---|
| 5X SuperScript II First Strand Buffer* | 4.0 μl | 4.0 μl | 6.0 μl | 8.0 μl | 10.0 μl | 20.0 μl |
| dNTP mix | 1.0 μl | 1.0 μl | 1.5 μl | 2.0 μl | 2.5 μl | 5.0 μl |
| 0.1 M DTT (supplied with enzyme) | 2.0 μl | 2.0 μl | 3.0 μl | 4.0 μl | 5.0 μl | 10.0 μl |
| Superscript II enzyme, 200 units | 1.0 μl | 1.0 μl | 1.5 μl | 2.0 μl | 2.5 μl | 5.0 μl |
| Superase-in RNase Inhibitor | 1.0 μl | 1.0 μl | 1.5 μl | 2.0 μl | 2.5 μl | 5.0 μl |
| Total Volume | 9.0 μl | 9.0 μl | 13.5 μl | 18.0 μl | 22.5 μl | 45.0 μl |

This is the Reaction Master Mix. (The Reaction Master Mix should be kept on ice until used).
5. Gently mix (do not vortex) and microfuge briefly to collect reaction mix contents in the bottom of the tube.
6. Add 4.5 μl of reaction mix from step 5 to the 6 μl of RNA-RT primer mix from step 1 (10.5 μl final volume).
7. Gently mix (do not vortex) and incubate at 42° C. for two hours.
8. Stop the reaction by adding 1.0 μl of 1.0M NaOH/100 mM EDTA.
9. Incubate at 65° C. for ten minutes to denature the DNA/RNA hybrids and degrade the RNA.
10. Neutralize the reaction with 1.2 μl of 2M Tris-HCl, pH 7.5. The cDNA synthesis preparation is now ready for use in an experiment (microarrays, blots, FISH, etc.), without any needed post-synthesis concentration. If desired, the cDNA preparation can be used with any post-synthesis purification as well.

### Example 2

### Method of cDNA Synthesis for Use with Microarrays without Post-Synthesis Sample Concentration

The following method is an example of a method for synthesis of cDNA for use with microarrays, wherein, in accordance with the present invention, the method of synthesis avoids the need for post-synthesis sample concentration. In the preferred embodiment disclosed herein, the method is designed for use with reagents from Genisphere, Inc., and in particular with the Genisphere® 3DNA Array 900 kit (available from Genisphere, Inc. of Montvale, N.J. and Hatfield Pa.), which is designed to provide increased sensitivity on microarrays when using extremely small quantities of RNA. (The instructions for use of the Array 900 kit provided with the kit are fully incorporated herein by reference). Alternatively, the method can be used with other kits or systems, using the method for small volume synthesis disclosed below.

In the preferred method, better sensitivity is achieved through the use of the modified cDNA synthesis protocol of the present invention that eliminates post-synthesis sample concentration, which avoids loss of sample during the concentration process. Sensitivity can also be further enhanced by use of a Genisphere® 3DNA Capture Reagent in the form of a dendrimer containing approximately 850 fluorescent dyes and engineered for more efficient hybridization kinetics, and by use of the Genisphere® 2× Enhanced cDNA Hybridization Buffer designed for better hybridization efficiency.

The Genisphere® 3DNA Array 900 kit is easy to use and is designed for use with arrays printed with oligonucleotides or PCR products (cDNA). First, either total or poly(A)+ RNA is reverse transcribed, using a deoxynucleotide triphosphate mix and special RT dT primer. Then, the cDNA and the fluorescent 3DNA reagent are hybridized to the microarray in succession. The fluorescent 3DNA reagent will hybridize to the cDNA because it includes a “capture sequence” that is complementary to a sequence on the 5′ end of the RT primer.

The 3DNA Array 900 labeling system provides a more predictable and consistent signal than direct or indirect dye incorporation for two reasons. First, since the fluorescent dye is part of the 3DNA reagent, it does not have to be incorporated during the cDNA preparation. This avoids inefficient hybridization of the cDNA to the array that results from the incorporation of fluorescent dye nucleotide conjugates into the reverse transcript. Second, because each 3DNA molecule contains an average of about 850 fluorescent dyes and each bound cDNA will be detected by a single 3DNA molecule, the signal generated from each message will be largely independent of base composition or length of the transcript. In contrast, the signal generated from each message labeled through dye incorporation will vary depending on the base composition or length of the message.

It should be noted that the array pattern produced by this kit may differ somewhat from the pattern produced by direct or indirect dye incorporation labeling methods when total RNA samples are used. The reason for this is that reverse transcriptase enzyme is known to label genomic DNA (without the need for a primer) as well as RNA. Dye incorporation labeling methods can therefore produce labeled genomic DNA. The labeled genomic DNA will bind to microarrays, resulting in false positives for negative genes and/or inappropriate and misleading fluorescence levels for array elements simultaneously bound with cDNA produced by reverse transcription of RNA. The 3DNA reverse transcription process, in contrast, utilizes unlabeled nucleotides that cannot incorporate any fluorescence into genomic DNA, thus eliminating the possibility of signal contribution from genomic DNA. Because 3DNA labeling differs from dye incorporation labeling in this way, the array pattern produced may vary depending on which labeling method is used. However, in a differential expression experiment, the expression patterns between the two RNA samples should be the same regardless of the labeling method, provided the genomic DNA has been eliminated from the samples.

Kit Contents

(Certain components of the Genisphere® Array 900 Kit, specifically Vials 1, 2 and 11, may not be compatible with other microarray labeling kits).

Vial 1 Cy3/Alexa Fluor 546 (red cap) or Cy5/Alexa Fluor 647 (blue cap) 3DNA Array 900 Capture Reagent. (Dendrimer probe reagents as described herein labeled with about 850 fluorescent dyes per molecule. Dendrimers labeled with Cy3 (or Alexa Fluor 546) contain the same unique capture sequence which targets the dendrimer only to cDNAs synthesized with primers containing 5 prime sequence complementary to the dendrimer bound capture sequence. Cy5 (or Alexa Fluor 647) labeled dendrimers contain a different capture sequence, allowing differential binding of Cy3 and Cy5 labeled dendrimers to cDNA).
Vial 2 1.0 pmole/μl RT Primer for Cy3/Alexa Fluor 546 (red cap) or Cy5/Alexa Fluor 647 (blue cap). (RT primers (48mers) comprising a 3 prime dT(17) and a 5 prime 31mer capture sequence).
Vial 3 Deoxynucleotide Triphosphate Mix (10 mM each dATP, dCTP, dGTP, and dTTP in water)
Vial 4 Superase-In™ RNase inhibitor (Ambion)
Vial 5 2× Enhanced cDNA Hybridization Buffer (available from Genisphere Inc. of Montvale, N.J. and Hatfield, Pa.)
Vial 6 2×SDS-Based Hybridization Buffer (0.50M NaPO4; 1% SDS; 2 mM EDTA; 2×SSC; 4×Denhardt's Solution)
Vial 7 2× Formamide-Based Hybridization Buffer (50% Formamide; 8×SSC; 1% SDS; 4×Denhardt's Solution)
Vial 8 Anti-Fade Reagent (0.1M DTT)
Vial 9 LNA TM dT Blocker (for use with PCR product (cDNA) microarrays) (a dT blocker comprising 36 nucleotides where 13 of the nucleotides are LNA (locked nucleic acid, Exiqon AG) residues. See e.g., U.S. Nonprovisional application Ser. No. 10/234,069 filed Sep. 3, 2002 which claims the priority of U.S. Provisional Application Ser. No. 60/316,116 filed Aug. 31, 2001, and “Methods for Blocking Nonspecific Hybridizations of Nucleic Acids”, International Application No. PCT/US02/027799 filed 3 Sep. 2002, International Publication No. WO 03/020902 A2, all of which are fully incorporated herein by reference).
Vial 10 Nuclease Free Water (available from Ambion)
Vial 11 5.0 pmole/μl RT Primer for Cy3/Alexa Fluor 546 (red cap) or Cy5/Alexa Fluor 647 (blue cap)
   (RT primers (48mers) comprising a 3 prime dT(17) and a 5 prime 31mer capture sequence)
      Vials 1-11 should be stored at −20° C. in the dark. Vial 1 may be kept at 4° C. for short-term storage (˜1 week).
      Other Materials Required
      Further materials required for use of this method include:
   A microarray: commercial or in-house prepared from either oligonucleotides or PCR/cDNA products
   A microarray reader equipped to read Cy3/Alexa Fluor 546 and/or Cy5/Alexa Fluor 647 fluorochromes
   Total RNA sample (greater than or equal to 100 ng/μl) or Poly(A)⁺ RNA sample (greater than or equal to 50 ng/μl)
   Reverse Transcriptase enzyme
   SuperScript II (Invitrogen Cat No. 18064-014-10,000 Units @ 200 U/μl)
   Genisphere RT Enzyme (Genisphere Cat No. RT300320)
   or other equivalent reverse transcriptase enzyme (Promega, etc).
   Cot-1 DNA (optional, species specific, available from Invitrogen)
   Reagent Grade Deionized Water (Recommended: VWR Cat No. RC9150-5)
   Note: As noted in the Internet List Serve, MilliQ® water has been shown to damage Cy5 (http://groups.yahoo.com/group/microarray/messages/2867).
   1.0M NaOH, 100 mM EDTA (cDNA synthesis stop solution)
   2M Tris-HCl, pH 7.5
   10 mM Tris-HCl, pH 8.0/1 mM EDTA (1×TE Buffer)
   Glass coverslips (Corning brand distributed by Fisher or VWR) or LifterSlipsÔ (Erie Scientific)
   2×SSC, 0.2% SDS buffer
   2×SSC buffer
   0.2×SSC buffer
   Glass Coplin Jars (or equivalent)
   0.5M NaOH/50 mM EDTA (optional: for use with Appendix A or B)
   1M Tris-HCl, pH 7.5 (optional: for use with Appendix A or B)
   Millipore Microcon®) YM-30 Centrifugal Filter Device (30,000 molecular weight cutoff, Millipore Cat No. 42409) (optional: for use with Appendix C—Millipore Microcon Procedure)
   Isopropanol (optional: for use with Appendix D)
   0.2% SDS buffer (optional: for use with Appendix D)
   95% ethanol (optional: for use with Appendix D)
   DyeSaver (Genisphere Cat No. Q100200) (optional: use to preserve fluorescent signal and prevent photobleaching)
      RNA Preparation:

Preparation and use of high-quality RNA is critical to the success of microarray experiments.

If degraded RNA is used, the RT reaction using dT primer will only generate short poly dT tails as opposed to full length cDNA, and little or no specific signal will be produced upon subsequent array hybridization. If degraded RNA samples must be used, good results can be obtained by labeling the samples with Genisphere's 3DNA Array 350RP (Version 2) kit.

The use of an RNase inhibitor (Superase-In, Vial 4) is strongly recommended. RNase inhibitor should be added to stored RNA samples suspected of being contaminated with RNases. Inhibitor should also be added during the reverse transcriptase reaction to avoid RNA degradation during cDNA synthesis. Please refer to the following references for more information regarding RNA degradation by RNases (all of which are fully incorporated herein by reference): Sambrook, J., Fritsch, E. F., and Maniatis, T. Molecular Cloning, A Laboratory Manual (Second Edition) Cold Spring Harbor Laboratory Press, 1989; Ausubel, F. M., Brent, R., Kingston, R. E., Moore, D. D., Seidman, J. G., Smith, J. A., and Struhl, K. Current Protocols in Molecular Biology. John Wiley & Sons, Inc., 1998.

The 3DNA Array 900 labeling system will not label genomic DNA, so it is not essential to remove genomic DNA contamination. However, the quantity and quality of the RNA present may be determined more accurately if the genomic DNA is digested away. Also, if the genomic DNA is allowed to remain in the sample, it may bind to some of the RT enzyme and make the enzyme unavailable for reverse transcription. RNase-free DNase is recommended for degrading contaminating genomic DNA.

If DNase is used, it is essential that the DNase be inactivated completely before proceeding with the cDNA synthesis procedure to prevent degradation of the RT Primer. Methods for inactivating DNase include phenol-chloroform extraction and use of the RNeasy® kits from Qiagen. Inactivation of the DNase by high temperature may not completely inactivate the enzyme.

High-quality RNA will have the following characteristics:
1. OD 260/280 ratio will be between 1.9 and 2.1.
2. On an agarose gel, total plant and mammalian RNA will be represented as two sharp, bright bands. For mammalian RNA, the bands will be at ˜4.5 kb and ˜1.9 kb, representing the 28S and 18S ribosomal sub-units, respectively.
   (Any suitable protocol known in the art for RNA purification to produce such high-quality RNA may be utilized, including, for example, those of Genisphere®).
   Selection and Preparation of Microarrays:

Pre-spotted cDNA arrays manufactured by Genomic Solutions, Agilent and Takara do not require special treatment prior to use. Pre-spotted oligo arrays manufactured by MWG Biotech require prewashing as described in Appendix E for optimal results. With other purchased arrays, prepare or pre-treat the microarray as described by the manufacturer. For arrays made “in-house”, it is recommended that one of the protocols in Appendix D be used for pre-treating the arrays. These protocols do not require succinic anhydride treatment and on many array types have yielded stronger signal and lower background. (Please note: This protocol is not compatible with Agilent arrays).

Genisphere recommends use of certain amino-silane coated slides for spotting PCR products (cDNAs), particularly Clontech DNA-Ready Type II Corning GAPS II and UltraGAPS, and Telechem SuperAmine slides. These slides demonstrate good DNA binding when used with 3DNA Array 900 kits.

Arrays prepared on poly-L-lysine, aldehyde or certain amino-silane (e.g., Corning GAPS) surfaces may require either a prewash or prehybridization step to reduce the background observed after hybridization. Refer to Appendix E or F, respectively, for procedures to help reduce background on microarrays.

As arrays age, they may exhibit lower specific signal and higher levels of background noise. In some cases, as an array ages the spotted probe demonstrates a “green” (Cy3) channel background. This has been experienced with both commercial and “in-house” arrays on all substrate surfaces. Quality control testing of both commercial and “in-house” arrays should be performed immediately after spotting (or receipt of arrays) and periodically thereafter to establish non-specific background noise characteristics of the arrays and other materials as they age. Also, all solutions used in post-spotting array processing should be tested to assure consistency and minimal contribution to non-specific array background.

Hybridization Conditions:

Because microarrays vary, it is important to determine the optimal hybridization conditions, including the optimal buffer selection, for each array type. The Genisphere Array 900 kit, for example, includes the following hybridization buffers:
1. 2× Enhanced cDNA Hybridization Buffer (Vial 5)—for use in the cDNA hybridization step only, if additional sensitivity is desired. This buffer may be used on arrays that tolerate higher temperatures (up to 65° C.).
2. 2×SDS-Based Hybridization Buffer (Vial 6)—for use in both the cDNA and 3DNA hybridization steps, this buffer may be used on arrays that tolerate higher temperatures (up to 65° C.).
3. 2× Formamide-Based Hybridization Buffer (Vial 7)—for use in both the cDNA and 3DNA hybridization steps, this buffer is designed for use at lower temperatures due to its higher stringency formulation.

It is recommended that the hybridization buffers be tested to determine which is best for the array type being utilized. Please note that due to the viscous nature of the 2× Enhanced cDNA Hybridization Buffer (Vial 5), a larger hybridization volume may be required under a coverslip due to volume loss when pipetting. Additionally, the ranges of hybridization temperatures included in this product manual are provided as a guide. Genisphere recommends that the optimal hybridization temperature be empirically determined for each lot of microarrays. For example, the poly-L-lysine surface of some arrays may begin to peel off at the hybridization temperature required for use of the Vial 5 or 6 buffer. Use Vial 7 buffer as directed if you experience this problem.

Because most PCR products contain poly(dA/dT) sequences, the use of the LNA dT Blocker (Vial 9) is recommended on cDNA arrays. The LNA dT Blocker is a high-performance poly T based blocking reagent designed by Genisphere (patent pending, see, International Application No. PCT/US02/027799 filed 3 Sep. 2002, International Publication No. WO 03/020902 A2, which is fully incorporated herein by reference). It is designed to completely block all poly A sequences present in array features, including control spots containing only poly dA sequences. This new blocking reagent contains Locked Nucleic Acid (LNA) nucleotides (a patented Exiqon™ technology) at key positions within the poly dT synthetic strand. The presence of these modified nucleotides stabilizes the hybridization between complementary strands of nucleic acids, thus improving the blocking capacity of the poly dT reagent. See reference 3 below for additional information relating to LNA chemistry. Although average array signal intensity for a blocked array may be lower compared to a non-blocked array, specific signal from reversed transcribed cDNA binding to complementary array elements should not be adversely affected. While a volume of 2 μl of the LNA dT Blocker (Vial 9) is recommended for each hybridization, some arrays may demonstrate better performance if additional LNA dT Blocker is used (3-4 μl).

Add additional competitor nucleic acid as required (e.g. species specific Cot-1 DNA from Invitrogen). Use competitor nucleic acid at 1/10 by mass of input total RNA (i.e. use 100 ng of Cot-1 DNA for every microgram of total RNA). If too much competitor is used, the signal may be reduced due to nonspecific interactions of the excess competitor with the limited cDNA in the hybridization. Denaturation of Cot-1 DNA and other competitor nucleic acids is recommended (95-10° C. for 5-10 minutes) prior to addition to the hybridization mix.

Procedure for Use

The following summarizes the steps necessary to synthesize cDNA from total RNA. If you are using poly(A)⁺ RNA, follow the procedure described in Appendix B for cDNA synthesis. Since microarrays and RNA preparations vary in quality, the exact amount of RNA required for a given experiment will typically range from 0.25-1.0 μg of animal total RNA or 0.5-2.5 μg of plant total RNA. For new users, 1 μg of animal total RNA or 2 μg of plant total RNA is recommended as a starting point for cDNA synthesis. Larger or smaller amounts of RNA may be required to achieve optimal results, depending on the quality of the RNA sample and the array.

Larger quantities of RNA (>2 μg) may be used for large-scale cDNA synthesis. Appendix A details a simple procedure for synthesizing cDNA from 2-50 μg total RNA. Alternatively, the procedure outlined on page 11 may be scaled up to accommodate larger quantities of total RNA. However, the larger reaction volume that results may require concentration of your cDNA samples (see Appendix C). Also, cDNA prepared using procedures from certain other Genisphere kits may also be used in conjunction with the Array 900 kit. The following Genisphere kits contain components for cDNA synthesis that generate cDNA compatible with the Array 900 3DNA Capture Reagents (Vial 1):
Array 350 (Catalog No. W300100, W300110, W300130, W300140, W300180, and W300184)
Array 350HS (Catalog No. H300100, H300110, H300130, H300140, H300180, and H300184)
Array 50 (Version 2) (Catalog No. B100121, B100122, B100171, B100172, B100187, and B100189)

### cDNA Synthesis from Total RNA

### Method for Small Volume Synthesis without Post-Synthesis Concentration

Please note: This procedure requires the pipetting of extremely small volumes of samples and reagents. The use of a pipetter designed for accurate and precise pipetting between 0.5 and 10 μl is recommended.
1. In a 0.5 mL “non-stick” microtube, prepare the RNA-RT primer mix:
   1-5 μl total RNA (0.25-1.0 μg mammalian total RNA or 0.5-2.5 μg plant total RNA)
   1 μl RT Primer (Vial 2 pmole/μl)
   Add Nuclease Free Water (Vial 10) to a final volume of 6 μl.
2. Mix the RNA-RT primer mix and microfuge briefly to collect contents in the bottom of the tube.
3. Heat to 80° C. for five minutes and immediately transfer to ice for 2-3 minutes. Microfuge briefly to collect contents in the bottom of the tube and return to ice.

4. Prepare a Reaction Master Mix in a microtube on ice (see table below). The Reaction Master Mix should be formulated to a final volume dependent on the number of cDNA syntheses set up simultaneously. Each cDNA synthesis requires 4.5 μl of Reaction Master Mix. To reduce pipetting errors, the Reaction Master Mix should contain at least 9 μl.

| Number of cDNA | | | | | | | | | | | | |
|---|---|---|---|---|---|---|---|---|---|---|---|---|
| syntheses | 1 | | 2 | | 3 | | 4 | | 5 | | 10 | |
| 5X SuperScript II | 4 | μl | 6 | μl | 8 | μl | 10 | μl | 12 | μl | 22 | μl |
| First Strand Buffer | | | | | | | | | | | | |
| 0.1 M DTT (supplied | 2 | μl | 3 | μl | 4 | μl | 5 | μl | 6 | μl | 11 | μl |
| with enzyme) | | | | | | | | | | | | |
| Superase-in RNAse | 1 | μl | 1.5 | μl | 2 | μl | 2.5 | μl | 3 | μl | 5.5 | μl |
| Inhibitor (Vial 4) | | | | | | | | | | | | |
| DNTP mix (Vial 3) | 1 | μl | 1.5 | μl | 2 | μl | 2.5 | μl | 3 | μl | 5.5 | μl |
| Superscript II enzyme, | 1 | μl | 1.5 | μl | 2 | μl | 2.5 | μl | 3 | μl | 5.5 | μl |
| 200 units | | | | | | | | | | | | |
| Reaction Master Mix | 9 | μl | 13.5 | μl | 18 | μl | 22.5 | μl | 27 | μl | 49.5 | μl |
| Total Volume | | | | | | | | | | | | |

Gently mix (do not vortex) and microfuge briefly to collect the Reaction Master Mix contents in the bottom of the tube. Keep on ice until ready to use.
5. Add 4.5 μl of the Reaction Master Mix from step 4 to the 6 μl of RNA-RT primer mix from step 3 (10.5 μl final volume).
6. Gently mix (do not vortex) and incubate at 42° C. for two hours.
7. Stop the reaction by adding 1.0 μl of 1.0M NaOH/100 mM EDTA.
8. Incubate at 65° C. for ten minutes to denature the DNA/RNA hybrids and degrade the RNA.
9. Neutralize the reaction with 1.2 μl of 2M Tris-HCl, pH 7.5.
10. Proceed to “Successive Hybridization of cDNA and 3DNA to Microarray” below.
   Successive Hybridization of cDNA and 3DNA to Microarray
   (Note: The following protocol is not compatible with Agilent arrays).
   cDNA Hybridization:
1. Thaw and resuspend the 2× Hybridization Buffer (Vial 5, Vial 6, or Vial 7) by heating to 65-70° C. for at least 10 minutes or until completely resuspended. Vortex to ensure that the components are resuspended evenly. If necessary, repeat heating and vortexing until all the material has been resuspended. Microfuge for 1 minute.
2. For each array, prepare the following cDNA Hybridization Mix for use with a 24×50 glass coverslip*:
   12.7 μl cDNA Synthesis #1
   12.7 μl cDNA Synthesis #2 or Nuclease Free Water (Vial 10) for single channel experiment
   27.4 μl 2× Hybridization Buffer (Vial 5, 6 or 7) (50% of the final cDNA Hybridization Mix)
   2 μl LNA dT Blocker (Vial 9) (may not be required for oligo arrays)
   54.8 μl total volume
      *Alternatively, if a smaller coverslip and hybridization volume is desired, simply prepare more cDNA than would be used on a single array and load less cDNA volume per array. For example, a 30 μl hybridization volume containing cDNA from 500 ng of total RNA per channel may be achieved by starting with 1000 ng of total RNA in the cDNA synthesis and using one half the volume of the final cDNA reverse transcription reaction. The final hybridization mix would contain:
   6.5 μl cDNA synthesis #1
   6.5 μl cDNA synthesis #2
   15.0 μl 2× Hybridization Buffer
   2.0 μl LNA Blocker
   30.0 μl total volume
   Optional: 1.0 μl C0T-1 DNA may also be added if desired (must be denatured at 95-100° C. for 10 minutes prior to use).
   If a larger coverslip or LifterSlip is required, the cDNA Hybridization Mix volume may be increased by adding equal volumes of 2× Hybridization Buffer (Vial 5, 6 or 7) and Nuclease Free Water (Vial 10).
   Note: Due to the viscous nature of the 2× Enhanced cDNA Hybridization Buffer (Vial 5), a larger hybridization volume may be required due to volume loss when pipetting. To address this, increase the total volume of the cDNA Hybridization Mix by adding equal volumes of Nuclease Free Water (Vial 10) and 2× Enhanced cDNA Hybridization Buffer (Vial 5).
   (Note: This product has not been validated for use in hybridization stations).
3. Gently vortex and briefly microfuge the cDNA Hybridization Mix after addition of all components. Incubate the Hybridization Mix first at 75-80° C. for 10 minutes, and then at the hybridization temperature until loading the array (see the table located below step 5 for recommended hybridization temperatures). Pre-warm the microarrays to the hybridization temperature.
4. Gently vortex and briefly microfuge the cDNA Hybridization Mix. Add the cDNA Hybridization Mix to a pre-warmed microarray, taking care to leave behind any precipitate at the bottom of the tube.

5. Apply the appropriate glass coverslip to the array. Incubate the array overnight in a dark humidified chamber at the appropriate hybridization temperature:

| Spotted DNA Size | Vial 5 or 6 Buffer | Vial 7 Buffer |
|---|---|---|
| 30 mer | 42-47° C. | 30-35° C. |
| 50 mer | 55-60° C. | 42-48° C. |
| 70 mer | 55-62° C. | 42-50° C. |
| PCR Products (cDNA) | 55-65° C. | 43-53° C. |

The hybridization temperatures recommended in this protocol are intended as a starting point and should be used as a guide. It may be necessary to adjust the temperatures to meet the stringency requirements dictated by the nature of the nucleic acids spotted on the array as well as the slide surface chemistry. In particular, increasing the hybridization temperature by 5° C. may remove non-specific signal.

Post cDNA Hybridization Wash:

1. Prewarm the 2×SSC, 0.2% SDS wash buffer:
   55-65° C. for PCR product (cDNA) arrays
   42° C. for oligonucleotide spotted arrays
2. Remove the coverslip by washing the array in prewarmed 2×SSC, 0.2% SDS for 2-5 minutes or until the coverslip floats off.* Additional time may be required to remove the coverslip when the 2× Enhanced cDNA Hybridization Buffer (Vial 5) is used.
   *Note: If the coverslip is difficult to remove, this may be an indication of drying. To prevent this problem from recurring in future experiments, increase the total volume of the cDNA Hybridization Mix by adding equal volumes of Nuclease Free Water (Vial 10) and 2× Hybridization Buffer (Vial 5, 6 or 7). In addition, ensure that the hybridization chamber is properly humidified and sealed.
3. Wash for 10-15 minutes in prewarmed 2×SSC, 0.2% SDS.
4. Wash for 10-15 minutes in 2×SSC at room temperature.
5. Wash for 10-15 minutes in 0.2×SSC at room temperature.
6. Transfer the array to a dry 50 mL centrifuge tube, orienting the slide so that any label is down in the tube. Immediately centrifuge without the tube cap for 2 minutes at 800-1000 RPM to dry the slide (any delay in this step may result in high background). Avoid contact with the array surface.
7. Further optimization of wash conditions may be required to achieve optimal array performance. If necessary to reduce background on the array, it is recommended that the time of some or all of the washes be increased to 15-20 minutes. Agitation during washing may also help to reduce background due to non-specific binding to the surface of the array.
   3DNA Hybridization:
   1. Prepare the 3DNA Array 900 Capture Reagent (Vial 1). It is necessary to break up aggregates that may form as a result of the freezing process.
   a. Thaw the 3DNA Array 900 Capture Reagent (Vial 1) in the dark at room temperature for 20 minutes.
   b. Vortex at the maximum setting for 3 seconds and microfuge briefly.
   c. Incubate at 50-55° C. for 10 minutes.
   d. Vortex at the maximum setting for 3-5 seconds.
   e. Microfuge the tube briefly to collect the contents at the bottom.
   Be sure to check the sample for aggregates prior to use and repeat vortex mixing if necessary. Aggregates may appear as small air bubbles or flakes at the side of the tube below the surface of the solution. Repeat steps a-e if necessary.
      2. Thaw and resuspend the 2× Hybridization Buffer (Vial 6 or Vial 7) by heating to 70° C. for at least 10 minutes or until completely resuspended. Vortex to ensure that the components are resuspended evenly. If necessary, repeat heating and vortexing until all the material has been resuspended. Microfuge for 1 minute.
   Caution: Do not use the 2× Enhanced cDNA Hybridization Buffer (Vial 5) in the 3DNA Hybridization step.
      3. The Anti-Fade Reagent (Vial 8) reduces fading of the fluorescent dyes post hybridization. Prepare a stock solution by adding 111 of Anti-Fade to 100 μl of 2× Hybridization Buffer (Vial 6 or Vial 7). Store any unused hybridization buffer at −20° C. and use within two weeks. However, do not use the Anti-Fade Reagent if your arrays are printed on aldehyde-coated slides, as background haze may result. Refreeze the Anti-Fade Reagent after use.
      4. For each array, prepare the following 3DNA Hybridization Mix for use with a 24×50 glass coverslip*:
   2.5 μl Cy3/Alexa Fluor S46 3DNA Array 900 Capture Reagent (Vial 1)
   2.5 μl Cy5/Alexa Fluor 647 3DNA Array 900 Capture Reagent (Vial 1)
   27.5 μl 2× Hybridization Buffer (Vial 6 or 7) (50% of the 3DNA Hybridization Mix)
   22.5 μl Nuclease Free Water (Vial 10)
   55.0 μl total volume
      *Alternatively, if a smaller coverslip and hybridization volume is desired, adjust the volume of the 2× Hybridization Buffer (Vial 6 or 7) and Nunclease Free Water (Vial 10) to a volume appropriate for the desired coverslip. For example, the final 3DNA Hybridization Mix for a 30 μl volume would contain:
      2.5 μl Cy3/Alexa Fluor 546 3DNA Array 900 Capture Reagent (Vial 1)
      2.5 μl Cy5/Alexa Fluor 647 3DNA Array 900 Capture Reagent (Vial 1)
      15.0 μl 2× Hybridization Buffer (Vial 6 or 7) (50% of the 3DNA Hybridization Mix)
      10.0 μl Nuclease Free Water (Vial 10)
      30.0 μl total volume
   Optional: 1.01 Cot-1 DNA may also be added if desired (must be denatured at 95-100° C. for 10 minutes prior to use).
   Note: For single channel expression analysis, use 2.5 μl of Nuclease Free Water (Vial 10) in place of the second 3DNA Array 900 Capture Reagent.
      5. Gently vortex and briefly microfuge the 3DNA Hybridization Mix. Incubate the 3DNA Hybridization Mix first at 75-80° C. for 10 minutes, and then at the hybridization temperature until loading the array (see the table located below step 7 for recommended hybridization temperatures).
      6. Gently vortex and briefly microfuge the 3DNA Hybridization Mix. Add the 3DNA Hybridization Mix to a pre-warmed microarray, taking care to leave behind any precipitate at the bottom of the tube. (Pre-warming the microarray to the hybridization temperature may reduce background.)

7. Apply a 24×50 glass coverslip to the array. If a larger coverslip or Lifterslip is required, the 3DNA Hybridization Mix volume may be increased by adding equal volumes of 2× Hybridization Buffer (Vial 6 or 7) and Nuclease Free Water (Vial 10). Incubate the array for 4-5 hours in a dark humidified chamber at the appropriate hybridization temperature:

| Spotted DNA Size | Vial 6 Buffer | Vial 7 Buffer |
|---|---|---|
| 30 mer | 55-65° C. | 45-53° C. |
| 50 mer | 55-65° C. | 45-53° C. |
| 70 mer | 55-65° C. | 45-53° C. |
| PCR Products (cDNA) | 60-65° C. | 50-55° C. |

Post 3DNA Hybridization Wash:
After hybridization the slides are washed several times to remove unbound 3DNA molecules. Perform these washes in the dark to avoid photobleaching and fading of the fluorescent dyes. To reduce fading of Cy5 post hybridization, it may also be beneficial to include DTT in the first two wash buffers at a final concentration of 0.5-1 mM. Be sure to work with fresh DTT, as old or poor quality DTT may cause an increase in background visible as a “haze” in the Cy3 channel. Please refer to Appendix G for recommendations for reducing the degradation of Cy5 when performing microarray experiments.
Caution: In the preparation of wash buffers, avoid the use of water that may cause damage Cy5/Alexa 647. As noted in the Internet List Serve, MilliQ® water has been shown to damage Cy5 (http://groups.yahoo.com/group/microarray/messages/2867). Also, be certain that any DEPC treated solutions have had all of the DEPC fully removed (DEPC is a potent oxidizer). Alternatively, the use of non-DEPC treated nuclease free solutions is recommended. Commercially available solutions (water, buffers, etc.) from Ambion have been found to work well with Cy5 labeled microarrays. In addition to Ambion water, DI water from VWR (Cat. No. RC91505) is also recommended. Water from Ambion and VWR have been validated for use with microarrays and do not contain components that will oxidize Cy5.
1. Prewarm the 2×SSC, 0.2% SDS wash buffer as follows:
   1. 65° C. for PCR product (cDNA) arrays and oligonucleotide arrays greater than 50 nucleotides long
   2. 42° C. for oligonucleotide arrays less than 50 nucleotides long
      8. Remove the coverslip by washing the array in prewarmed 2×SSC, 0.2% SDS for 2-5 minutes or until the coverslip floats off.*
      *Note: If the coverslip is difficult to remove, this may be an indication of drying. To prevent this problem from recurring in future experiments, increase the total volume of the 3DNA Hybridization Mix by adding equal volumes of Nuclease Free Water (Vial 10) and 2× Hybridization Buffer (Vial 6 or 7). In addition, ensure that the hybridization chamber is properly humidified and sealed.
      9. Wash for 10-15 minutes in prewarmed 2×SSC, 0.2% SDS.
      10. Wash for 10-15 minutes in 2×SSC at room temperature.
      11. Wash for 10-15 minutes in 0.2×SSC at room temperature.
      12. Transfer the array to a dry 50 mL centrifuge tube, orienting the slide so that any label is down in the tube. Immediately centrifuge without the tube cap for 2 minutes at 800-1000 RPM to dry the slide (any delay in this step may result in high background). Avoid contact with the array surface.
      Further optimization of wash conditions may be required to achieve optimal array performance. If necessary to reduce background on the array, increase the time of some or all of the washes to 15-20 minutes. Agitation during washing may also help to reduce background due to non-specific binding to the surface of the array.
      Proceed to “Signal Detection” or first apply DyeSaver coating (Genisphere Cat No. Q100200) to preserve fluorescent signal.
      Signal Detection
      IMPORTANT: Store the array in the dark until scanned. The fluorescence of the 3DNA reagents, especially Cy5 and Alexa Fluor647, can diminish rapidly even in ambient light because of oxidation. Please refer to Appendix F for recommendations for reducing the degradation of Cy5/Alexa Fluor 647 when performing microarray experiments.
      Scan the microarray as described by the scanner's manufacturer. Avoid excess multiple scans as the dyes may photobleach from exposure to the scanner light source.
      If using a Packard scanner, the recommendation is to start by setting the laser at 80% power and either use the “autobalance” feature or the table below to set up the initial scanning parameters for proper channel balance. Adjustment of your scanner laser power and photo-multiplier tube (PMT) voltage may be required to balance the various fluorophore channels. If the PMT setting is set too high, the background observed may be unacceptable. In these insane the PMT setting should be reduced and the laser power should be increased to optimize the signal-to-noise ratio. However, to prevent photobleaching the fluorescent dyes, especially Cy5/Alexa Fluor 647, after a single scan, avoid setting the laser too high (>90-95% power).

Note: Balancing the image by offsetting the laser or PMT may result in a non-linear distribution of the data between each channel. In these instances, a statistical normalization may be required. Please consult the instrument's user manual for further instructions.

Initial Scanner Setting
for Packard ScanArray 5000 4 Channel Scanner

| Dye | Laser | PMT |
|---|---|---|
| Cy3/Alexa Fluor 546 | 80 | 70 +/− 5 |
| Cy5/Alexa Fluor 647 | 80 | 65 +/− 5 |

If using an Axon 4000 series scanner, the recommended PMT settings are as follows:

Initial Scanner Setting for Axon 4000B 2 Channel Scanner

| Dye | Laser | PMT |
|---|---|---|
| Cy3/Alexa Fluor 546 | 100 | 500-700 volts |
| Cy5/Alexa Fluor 647 | 100 | 600-800 volts |

The following references provide further background as the techniques discussed herein, and are fully incorporated herein by reference:
1. Nilsen, T. W., Grayzel, J., and Prensky, W. Dendritic Nucleic Acid Structures. J. Theor. Biol. (1997) 187: 273-284.
2. Stears, R. L., Getts, R. C., Gullans, S. R. A novel, sensitive detection system for high-density microarrays using dendrimer technology. Physiol Genomics 3: 93-99, 900.
3. Singh, S. K., Nielsen, P., Koshkin, A. A., and Wengel, J. LNA (Locked Nucleic Acids): Synthesis and high-affinity nucleic acid recognition. Chem. Commun., 455-456, 1998.
   Note: Cy is a trademark of Amersham Biosciences; Alexa Fluor is a trademark of Molecular Probes; RNeasy and Qiagen are trademarks of Qiagen Inc.; Superase-In is a trademarked product of Ambion, Inc.; Exiqon and LNA are trademarks of Exiqon A/S; Millipore, MilliQ and Microcon are trademarks of Millipore, Inc.; LifterSlip is a trademark of Erie Scientific Co.; and, 3DNA, Genisphere, Array 350RP, Array 350, Array 50, Array 900 and DyeSaver are trademarks of Datascope Corp. of Montvale, N.J.

### APPENDIX A

Scaled-Up cDNA Preparation

A scaled up reverse transcription reaction can be performed from 2-50 μg of total RNA to provide extra cDNA for duplicate experiments, quantitation of the cDNA, or other parallel analysis.

cDNA Synthesis:

1. In a 0.5 or 1.5 mL RNase-free microcentrifuge tube, prepare the RNA-RT primer mix:
   1-10 μl total RNA (2-50 μg mammalian total RNA or 25-125 μg plant total RNA)
   1 μl RT Primer (Vial 11, 5 pmole/μl)
   Add RNase free water to a final volume of 11 μl
2. Mix the RNA-RT primer mix and microfuge briefly to collect contents in the bottom of the tube.
3. Heat to 80° C. for ten minutes and immediately transfer to ice for 2-3 minutes.
4. In a separate microtube combine (on ice):
   4 μl 5× Superscript II First Strand Buffer or equivalent reaction buffer (supplied with enzyme)
   2 μl 0.1M dithiotreitol (DTT) (supplied with the enzyme)
   1 μl Superase-In (Vial 4)
   1 μl dNTP mix (Vial 3)
   1 μl Superscript II enzyme, 200 Units

This is the reaction mix. The final volume should be 9 μl. Gently mix (do not vortex) and microfuge briefly to collect contents in the bottom of the tube. Keep on ice until used.
5. Add the 9 μl of reaction mix from step 4 to the 11 μl of RNA-RT primer mix from step 3 (20 μl final volume).
6. Gently mix (do not vortex) and incubate at 42° C. for two hours.
7. Stop the reaction by adding 3.5 μl of 0.5M NaOH/50 mM EDTA.
8. Incubate at 65° C. for ten minutes to denature the DNA/RNA hybrids and degrade RNA.
9. Neutralize the reaction with 5 μl of 1M Tris-HCl, pH 7.5. The resulting solution is your cDNA.
10. Dilute the cDNA to a concentration appropriate for the desired hybridization volume.
Proceed to “Successive Hybridization of cDNA and 3DNA to Microarray”.

### APPENDIX B

cDNA Preparation from Poly(A)⁺ RNA

The procedure below summarizes the steps necessary to synthesize cDNA from poly(A)⁺ RNA. Since microarrays and RNA preparations vary in quality, the exact amount of RNA required for a given experiment will range from 12.5-50 ng of poly(A)⁺ RNA. For new users, 50 ng of poly(A)⁺ RNA is recommended as a starting point for cDNA synthesis.

cDNA Synthesis:

1. In a 1.5 mL RNase-free microcentrifuge tube combine:
   1-10 μl poly(A)⁺ RNA (25-100 ng)
   1 μl RT Primer (Vial 11, 5 pmole(μl)
   Add RNase free water to a final volume of 11 μl
   This is the RNA-RT primer mix.
2. Mix and microfuge briefly to collect contents in the bottom of the tube.
3. Heat to 80° C. for ten minutes and immediately transfer to ice for 2-3 minutes.
4. In a separate microtube combine (on ice):
   4 μl 5× Superscript II First Strand Buffer or equivalent reaction buffer (supplied with enzyme)
   2 μl 0.1M dithiotreitol (DTT) (supplied with the enzyme)
   1 μl Superase-In (Vial 4)
   1 μl dNTP mix (Vial 3)
   1 μl Superscript II enzyme, 200 Units
This is the reaction mix. The final volume should be 9 μl. Gently mix (do not vortex) and microfuge briefly to collect contents in the bottom of the tube. Keep on ice until used.
5. Add the 9 μl of reaction mix from step 4 to the 11 μl of RNA-RT primer mix from step 3 (20 μl final volume).
6. Gently mix (do not vortex) and incubate at 42° C. for two hours.
7. Stop the reaction by adding 3.5 μl of 0.5M NaOH/50 mM EDTA.
8. Incubate at 65° C. for ten minutes to denature the DNA/RNA hybrids and degrade RNA.
9. Neutralize the reaction with 5 μl of 1M Tris-HCl, pH 7.5. The resulting solution is your cDNA.
10. Dilute the cDNA to a concentration appropriate for the desired hybridization volume.
Proceed to “Successive Hybridization of cDNA and 3DNA to Microarray”.

### APPENDIX C

Concentration of cDNA

cDNA samples may be concentrated using the Millipore Microcon YM-30 Centrifugal Filter Devices (30,000 molecular weight cutoff, Millipore catalog number 42409). These devices are capable of reducing the volume of the cDNA synthesis reaction to 3-10 μl in approximately 8-10 minutes. The procedure below reiterates the manufacturer's directions with minor adaptations for the 3DNA Array 900 Kit.

Caution: Use of the Millipore Microcon YM-30 Centrifugal Filter Devices may result in significant loss of small samples of cDNA (<1.0 μg).

Important: Users of the Microcon YM-30's should evaluate their own centrifuge settings to determine the optimal time and speed settings to yield final volumes of 3-10 μl.

1. Place the Microcon YM-30 sample reservoir into the 1.5 mL collection tube.
2. Pre-wash the reservoir membrane by adding 100 μl 1×TE buffer to the Microcon YM-30 sample reservoir.
3. Place the tube/sample reservoir assembly into a fixed angle rotor tabletop centrifuge capable of 10-14,000 g.
4. Spin for 3 minutes at 10-14,000 g.
5. Bring the volume of the cDNA reaction to 100 μl with 1×TE buffer. Add all of the cDNA reaction to the Microcon YM-30 sample reservoir. Do not touch the membrane with the pipet tip.
6. Place the tube/sample reservoir assembly into a fixed angle rotor tabletop centrifuge capable of 10-14,000 g.
7. Centrifuge for 8-10 minutes at 10-14,000 g.
8. Remove the tube/sample reservoir assembly. Separate the collection tube from the sample reservoir with care, avoiding spilling any liquid in the sample reservoir.
9. Add 5 μl of 1×TE buffer to the sample reservoir membrane without touching the membrane. Gently tap the side of the concentrator to promote mixing of the concentrate with the 1×TE buffer.
10. Carefully place the sample reservoir upside down on a new collection tube. Centrifuge for 2 minutes at top speed in the same centrifuge.
11. Separate the sample reservoir from the collection tube and discard the reservoir. Note the volume collected in the bottom of the tube (3-10 μl total volume). The cDNA sample may be stored in the collection tube for later use.
12. Add water to achieve the desired volume of cDNA.
   Proceed to “Successive Hybridization of cDNA and 3DNA to Microarray”.

### APPENDIX D

Array Processing Procedure (No Succinic Anhydride)

Option 1 (Recommended) (Cross-Link, Isopropanol Wash, and Boil):

1. Preheat 2 liters of reagent grade deionized distilled water (best quality water available) to 95° C.-100° C. (boiling) in a 4 liter beaker on a hot plate.
2. Transfer 250 mL of isopropanol into a glass rectangular staining dish and place a small stir bar into the dish. Place the dish on a magnetic stir plate and allow the bar to stir at a slow steady rate.
3. Retrieve the unprocessed arrays. Carefully, pick up one slide by the corner and hold it in the steam above the boiling water (from step 1) for five seconds. Make sure the arrays are facing up. Wave the slide in the air for three seconds and place onto a fiber free lab wipe, array side up. Repeat until eight slides have been hydrated and dried
4. Transfer the eight slides, array side up, to a cross-linker set to 50-220 mJ.
5. After cross-linking, transfer the eight arrays into a glass/metal Wheaton staining slide holder with grooves (do not place slides on each end groove). Put the holder with the slides into the isopropanol (from step 2) and incubate for 15 minutes with stirring.
6. Transfer the slide holder to the boiling water (from step 1) and incubate for 8-10 minutes. Make sure the slides are under the water.
7. Remove the slide holder from the boiling water and place onto a lab wipe to remove excess liquid. The arrays are now ready for hybridization.
   Option 2 (Cross-Link, SDS Wash, Boil, and Cold Ethanol Rinse):
1. Prepare 2 liters of a 0.2% SDS solution in reagent grade deionized distilled water (best quality water). For example, mix 40 mL of 10% SDS and 1960 mL of water in a two liter autoclaved glass bottle. Filter the solution to remove any precipitated SDS. Transfer 250 mL of this 0.2% SDS solution into a glass rectangular staining dish and place a small stir bar into the dish. Place the dish on a magnetic stir plate and allow the bar to stir at a slow steady rate.
2. Preheat 2 liters of reagent grade deionized distilled water (best quality water available) to 95° C.-100° C. (boiling) in a 4 liter beaker on a hot plate.
3. Transfer 2 liters of reagent grade deionized distilled water to a 4 liter beaker. Keep at room temperature.
4. Transfer 250 mL of ethanol into a glass rectangular staining dish. Place this dish into an ice bucket to set up an ice cold ethanol bath.
5. Retrieve the unprocessed arrays. Carefully, pick up one slide by the corner and hold it in the steam above the boiling water (from step 2) for five seconds. Make sure the arrays are facing up. Wave the slide in the air for three seconds and place onto a fiber free lab wipe, array side up. Repeat until eight slides have been hydrated and dried.
6. Transfer the eight slides, array side up, to a cross-linker set to 50-220 mJ.
7. After cross-linking, transfer the eight arrays into a glass/metal Wheaton staining slide holder with grooves (do not place slides on each end groove). Put the holder with the slides into the 0.2% SDS (from step 1) and incubate for 10 minutes with stirring.
8. Remove the slide holder and place onto a lab wipe to remove excess liquid. Then dunk the holder into the 2 liters of room temperature water (from step 3) five times.
9. Transfer the slide holder to the boiling water (from step 2) and incubate for 8-10 minutes. Make sure the slides are under the water.
10. Remove the slide holder from the boiling water and place onto a lab wipe to remove excess liquid. Transfer the slide holder into the ice cold ethanol (from step 4) and incubate for five minutes. Make sure the slides are under the ethanol.
11. Remove the slide holder and place onto a lab wipe to remove excess liquid. Transfer each slide into a 50 mL centrifuge tube. Centrifuge at 1000 rpm for 3 minutes to dry the slides. The arrays are now ready for hybridization.

### APPENDIX E

Array Prewashing Procedure to Reduce Background

1. Wash the microarray by the following conditions:
   a. 2×SSC/0.2% SDS for 20 minutes at 55° C.
   b. 0.2×SSC for 5 minutes at room temperature
   c. deionized distilled water for 3 minutes at room temperature
2. Immediately transfer the array to a dry 50 mL centrifuge tube. Do this quickly to avoid streaky background on the slide. Orient the slide so that any label is down in the tube. Centrifuge without the tube cap for 2 minutes at 800-1000 RPM to dry the slide. Avoid contact with the array surface. Transfer the microarray array to dish or Coplin jar with 0.2×SSC at room temperature for 5 minutes.
   The array is now ready for either prehybridization or hybridization with cDNA.

### APPENDIX F

Array Prehybridization to Reduce Background:

Non-specific binding to the array surface is a common problem on many array types. The prehybridization protocol described below is recommended for reducing some types of non-specific binding, thereby reducing the background seen post-hybridization.

1. Prewarm the microarray to 50° C. for 10 minutes.

2. Thaw and resuspend the 2× Hybridization Buffer (Vial 7) by heating to 70° C. for at least 10 minutes or until completely resuspended. Vortex to ensure that the components are resuspended evenly. If necessary, repeat heating and vortexing until all the material has been resuspended. Microfuge for 1 minute.

3. Prepare Prehybridization Mix as follows:

25 μl 2× Formamide-Based Hybridization Buffer (Vial 7)
1 μl Human Cot-1 DNA
24 μl Nuclease free water
   4. Heat the Prehybridization Mix to 80° C. for 10 minutes.
   5. Apply the Prehybridization Mix to the prewarmed microarray and cover with a 24×60 mm coverslip.
   6. Incubate at 50° C. for 1-2 hours.
   7. Wash the array by the following conditions:
a. 2×SSC, 0.2% SDS for 15 min at 60-65° C.
b. 2×SSC for 10 min at room temperature.
c. 0.2×SSC for 10 min at room temperature.
   8. Immediately transfer the array to a dry 50 mL centrifuge tube. Do this quickly to avoid streaky background on the slide. Orient the slide so that any label is down in the tube. Centrifuge without the tube cap for 2 minutes at 800-1000 RPM to dry the slide. Avoid contact with the array surface.

The array is now ready for hybridization with cDNA.

### APPENDIX G

Recommendations for Reducing the Degradation of Cy5 or Alexa Fluor 647 when Performing Microarray Experiments

Cy5/Alexa Fluor 647 dye performance may be affected by a variety of factors that are particularly prevalent during the summer months. Exposure of the Cy5/Alexa Fluor 647 dye solutions and the hybridized arrays to light and to oxidative environments may cause rapid fading of the Cy5/Alexa Fluor 647 dye, regardless of the labeling system used. Limiting or controlling the exposure of the arrays to these environments has been shown to significantly reduce Cy5/Alexa Fluor 647 fading.

Below are recommendations for reducing the degradation of Cy5/Alexa Fluor 647 when performing microarray experiments:

1. Always keep solutions and arrays containing Cy5/Alexa Fluor 647 away from light, particularly sunlight Cy5/Alexa Fluor 647 will photobleach when exposed to light, including normal fluorescent lighting.

2. Protect the hybridized, dried array from contact with air, particularly on hot and sunny days. Ambient ozone levels resulting from summertime air pollution can cause oxidative degradation Cy5/Alexa Fluor 647. Keeping the Cy5/Alexa Fluor 647-containing arrays in an inert atmosphere (nitrogen) in a small container (50mL tube) can significantly delay fading of the Cy5/Alexa Fluor 647. Some investigators also add small quantities of DTT or beta mercapto-ethanol (BME) to the bottom of the tube to further promote a reducing micro-environment (be certain to avoid contact of the array with these chemicals).

3. Use the Anti-Fade Reagent (provided with the 3DNA kits) in the hybridization solution containing Cy5/Alexa Fluor 647 Capture Reagent. The Anti-Fade Reagent has anti-oxidant properties that will retard the oxidative process.

4. In the preparation of wash buffers, avoid the use of water that may cause damage Cy5/Alexa 647. As noted in the Internet List Serve, MilliQ® water has been shown to damage Cy5 (http://groups.yahoo.com/group/microarray/messages/2867). Also, be certain that any DEPC treated solutions have had all of the DEPC fully removed (DEPC is a potent oxidizer). Alternatively, the use of non-DEPC treated nuclease free solutions is recommended. Commercially available solutions (water, buffers, etc.) from Ambion have been found to work well with Cy5 labeled microarrays. In addition to Ambion water, DI water from VWR (Cat. No. RC91505) is also recommended. Water from Ambion and VWR have been validated for use with microarrays and do not contain components that will oxidize Cy5.

5. Add a small quantity of dithiothreotol (DTT) to the post hybridization wash buffers, i.e. 0.1 mM final concentration. This potent reducing agent will protect the Cy5/Alexa Fluor 647 on the array from attack by any oxidative agents in the wash buffers.

6. Always be certain to mix your 3DNA Cy3/Alexa Fluor 546 and Cy5/Alexa Fluor 647 Capture Reagents (Vial 1) to break up any aggregates that may form during storage:

a. Thaw the 3DNA Capture Reagent (Vial 1) in the dark at room temperature for 20 minutes.
b. Vortex at the maximum setting for 3 seconds and microfuge briefly (1 second).
c. Incubate at 50-55° C. for 10 minutes.
d. Vortex at the maximum setting for 3-5 seconds.
e. Microfuge the tube briefly to collect the contents at the bottom.
Be sure to check the sample for aggregates prior to use and repeat vortex mixing if necessary. Aggregates may appear as small air bubbles or flakes at the side of the tube below the surface of the solution. Repeat steps a-e if necessary.
   7. If the above recommendations do not eliminate Cy5 degradation problems, the likely cause is exposure to atmospheric pollutants. To address this issue, Genisphere has developed a reagent, DyeSaver (Genisphere cat. no. Q100200), that is applied to the array after the final wash and spin. DyeSaver is easy to use, compatible with most array surface chemistries, and protects Cy5 from atmospheric oxidation for up to three weeks. DyeSaver has also been shown to reduce Cy5 damage due to photobleaching.

Having described this invention with regard to specific embodiments, it is to be understood that the description is not meant as a limitation since further embodiments, modifications and variations may be apparent or may suggest themselves to those skilled in the art. It is intended that the present application cover all such embodiments, modifications and variations.

1. A method comprising:
taking an initial sample of RNA;
reverse transcribing the sample of RNA to synthesize a sample of cDNA;
and,
applying the sample of cDNA to a microarray;
wherein the sample of cDNA is applied to the microarray without concentration of the sample of cDNA after the synthesis of the cDNA and before application of the sample of cDNA to the microarray.

2. A method as claimed in claim 1, wherein the sample of cDNA is applied to the microarray without purification of the sample of cDNA after synthesis of the sample of cDNA and before application of the sample of cDNA to the microarray.

3. A method as claimed in claim 1, wherein the initial sample of RNA comprises total RNA.

4. A method as claimed in claim 1, wherein the initial sample of RNA comprises messenger RNA.

5. A method as claimed in claim 1, wherein the initial sample of RNA comprises from approximately 0.1 to 1 micrograms of total RNA.

6. A method as claimed in claim 1, wherein the initial sample of RNA comprises from approximately 1 to 1000 nanograms of mRNA.

7. A method as claimed in claim 1, further comprising the step of hybridizing a dendrimer to the cDNA in the sample of cDNA.

8. A method as claimed in claim 1, further comprising the step of hybridizing a dendrimer to the cDNA in the sample of cDNA, wherein the dendrimer is labeled to produce a detectable signal.

9. A method as claimed in claim 1, further comprising the step of hybridizing a dendrimer to the cDNA in the sample of cDNA, wherein the dendrimer is labeled to produce a detectable signal, and wherein the dendrimer is labeled with greater than 500 fluorescent dyes on each dendrimer molecule.

10. A method comprising:
taking an initial sample of RNA;
reverse transcribing the sample of RNA to synthesize a sample of cDNA;
applying the sample of cDNA to a microarray, wherein the microarray has nucleic acid samples affixed thereto;
allowing the cDNA in the sample of cDNA to hybridize to the nucleic acid samples on the microarray; and,
hybridizing a dendrimer to the cDNA in the sample of cDNA;
wherein the sample of cDNA is applied to the microarray without concentration of the sample of cDNA after the synthesis of the cDNA and before application of the sample of cDNA to the microarray.

11. A method as claimed in claim 10, wherein the sample of cDNA is applied to the microarray without purification of the sample of cDNA after synthesis of the sample of cDNA and before application of the sample of cDNA to the microarray.

12. A method as claimed in claim 10, wherein the initial sample of RNA comprises total RNA.

13. A method as claimed in claim 10, wherein the initial sample of RNA comprises messenger RNA.

14. A method as claimed in claim 10, wherein the initial sample of RNA comprises from approximately 0.1 to 1 micrograms of total RNA.

15. A method as claimed in claim 10, wherein the initial sample of RNA comprises from approximately 1 to 1000 nanograms of mRNA.

16. A method as claimed in claim 10, further comprising the step of hybridizing a dendrimer to the cDNA in the sample of cDNA, wherein the dendrimer is labeled to produce a detectable signal.

17. A method as claimed in claim 10, further comprising the step of hybridizing a dendrimer to the cDNA in the sample of cDNA, wherein the dendrimer is labeled to produce a detectable signal, and wherein the dendrimer is labeled with greater than 500 fluorescent dyes on each dendrimer molecule.
